(19) United States
(12) Patent Application Publication
(10) Pub. No.: US 2008/0300527 A1
(43) Pub. Date: Dec. 4, 2008

(54) HANDHELD DEVICES FOR MANUALLY CLEANING BODY ORIFICES

(76) Inventor: Bruce R. Bivins, Des Moines, WA (US)

Correspondence Address: PERKINS COIE LLP;PATENT-SEA, P.O. BOX 1247, SEATTLE, WA, 98111-1247, US

(21) Appl. No.: 11/757,890

(22) Filed: Jun. 4, 2007

(51) Int. Cl. A61F 13/38; A61F 15/00; A61M 35/00

(52) U.S. Cl. 604/1; 15/176.2; 15/209.1

(57) ABSTRACT

Disposable handheld devices for manually cleaning nostrils and/or other body orifices are disclosed herein. A device configured in accordance with one embodiment of the invention includes a shaft having a first end portion spaced apart from a second end portion to define a first exterior surface portion therebetween with at least one ridge portion extending outwardly therefrom. The device can further include a first tip portion positioned toward the first end portion of the shaft and a second tip portion positioned toward the second end portion of the shaft. The first tip portion can include a second ridge portion extending outwardly from a second exterior surface portion. The second tip portion can include a third ridge portion extending outwardly from a third exterior surface portion.

### TECHNICAL FIELD

The present invention is directed generally towards personal cleaning devices that can be used to manually clean body orifices.

### BACKGROUND

Swabs for cleaning ears are well known. For example, U.S. Pat. No. 5,766,143 describes a cotton swab for cleaning the ear or applying cosmetics. Such swabs, however, may not be useful for cleaning other body orifices, such as nostrils. For example, such swabs may not be effective at removing dust or debris from nostrils.

U.S. Pat. No. 6,277,090 describes a nose-cleaning system comprising a handle and a removable foam tip. In this system, the tips are not intended to be disposable. As a result, dust and debris can accumulate on the tips over time, necessitating periodic cleansing.

Accordingly, there is a need for devices that effectively remove dust and other debris from nostrils and other body orifices.

### SUMMARY

The following summary is provided for the benefit of the reader only, and is not intended to limit in any way the invention as set forth by the claims. The present invention is directed generally to disposable handheld devices for manually cleaning nostrils and other body orifices. A handheld device configured in accordance with one aspect of the invention includes a shaft having a first end portion spaced apart from a second end portion to define a first exterior surface portion therebetween. The shaft can have a first ridge portion extending outwardly from the first exterior surface portion. The device can further include a first tip portion positioned toward the first end portion of the shaft. The first tip portion can have a second exterior surface portion defining a first generally conical shape. The first tip portion can further include a second ridge portion extending outwardly from the second exterior surface portion. The device can additionally include a second tip portion positioned toward the second end portion of the shaft. The second tip portion can have a third exterior surface portion defining a second generally conical shape. The second tip portion can further include a third ridge portion extending outwardly from the third exterior surface portion.

In another aspect of the invention, the first ridge portion can describe a generally helical path around the first exterior surface portion of the shaft. Similarly, the second ridge portion can describe a generally helical path around the second exterior surface portion of the first tip portion. Similarly, the third ridge portion can describe a generally helical path around the third exterior surface portion of the second tip portion.

In a further aspect of the invention, the handheld device can include a scented medium applied to the first and second tip portions. The scented medium can include an herbal scent, such as eucalyptus, tea tree oil, peppermint, etc. The scented medium can include other scents or fragrances to enhance the user experience. The first and second tip portions can also include cotton bodies composed of, for example, organic cotton. In other embodiments, the first and second tip portions can include compressible foam bodies, or other suitable materials known in the art.

### BRIEF DESCRIPTION OF THE DRAWINGS

FIG. 1 is an isometric view illustrating one use of a personal cleaning device in accordance with an embodiment of the invention.

FIG. 2 is an enlarged side view of a disposable, handheld personal cleaning device configured in accordance with an embodiment of the invention.

FIG. 3 is an isometric view of a disposable, handheld personal cleaning device configured in accordance with another embodiment of the invention.

FIG. 4 is an exploded, partially cut-away view of a portion of the disposable, handheld personal cleaning device of FIG. 3.

FIGS. 5A-5C are enlarged side views of personal cleaning device tip portions configured in accordance with embodiments of the invention.

FIGS. 6A-6C are enlarged side views of personal cleaning device tip portions configured in accordance with additional embodiments of the invention.

FIGS. 7A-7D are enlarged side views of personal clean device shaft portions configured in accordance with embodiments of the invention.

FIGS. 8A-8F are enlarged side views of personal cleaning device tip portions configured in accordance with embodiments of the invention

### DETAILED DESCRIPTION

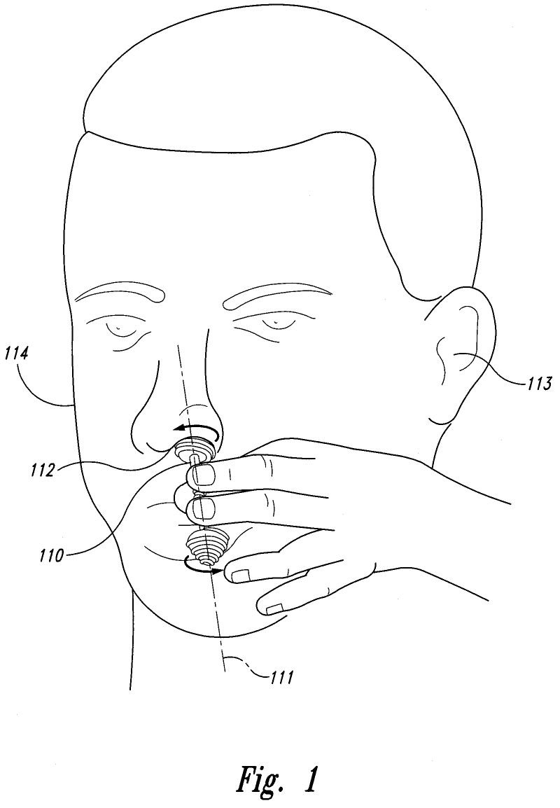

The present disclosure describes various types of handheld devices for manually cleaning nostrils and other body orifices. Specific details of the invention are set forth in the following description and in FIGS. 1-7D to provide a thorough understanding of certain embodiments of the invention. One skilled in the relevant art, however, will understand that the present invention may have additional embodiments, and that other embodiments of the invention may be practiced without several of the specific features described below.

FIG. 1 is an isometric view of a user 114 cleaning a nostril 112 with a personal cleaning device 110 configured in accordance with an embodiment of the invention. As shown in FIG. 1, the user 114 can insert the personal cleaning or personal hygiene device 110 into the nostril 112 and rotate the device 110 about its longitudinal axis 111 to loosen and/or remove debris from the nostril 112. The user 114 can also manipulate the device 110 in a variety of other ways, such as by moving the device 110 in an up-and-down fashion, a side-to-side fashion, etc. The user 114 can also use the device 110 to clean other body orifices, such as an ear canal 113.

The device 110 is appropriately sized and shaped for insertion into the nostril 112 of the user 114. As described in greater detail below, the device 110 has two tip portions so that the user 114 can clean both nostrils. Because the device 110 is disposable, the user 114 does not need to clean the tip portions after use.

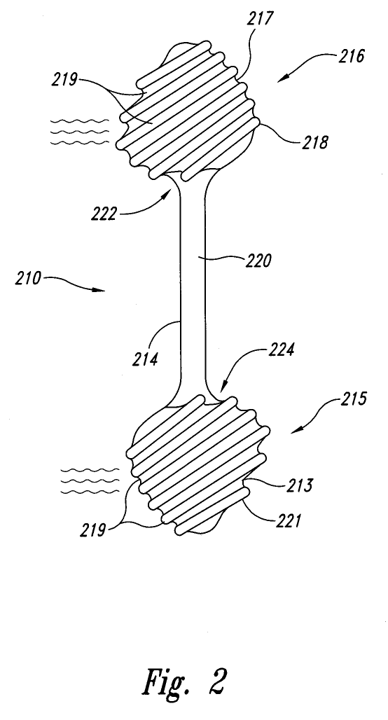

FIG. 2 is an enlarged side view of a personal cleaning device 210 configured in accordance with an embodiment of the invention. In one aspect of this embodiment, the device 210 includes a handle or shaft 220 having a first end portion 222 spaced apart from a second end portion 224 to define a first exterior surface portion 214 therebetween. A user (not shown) can grip the first exterior surface portion 214 to manipulate the device 210 during use.

A first tip portion 216 is attached toward the first end portion 222 of the shaft 220. In the illustrated embodiment, the first tip portion 216 has a second exterior surface portion 217 defining a first generally conical shape. The first tip portion 216 also has a first ridge portion 218 extending outwardly from the second exterior surface portion 217.

A second tip portion 215 is attached toward the second end portion 224 of the shaft 220. The second tip portion 215 has a third exterior surface portion 213 defining a second generally conical shape. The second tip portion 215 also has a second ridge portion 221 extending outwardly from the third exterior surface portion 219. The generally conical shape of the first tip portion 216 and the second tip portion 215 can prevent a user from inserting the device 210 too far into a nostril or other body orifice.

In addition to the foregoing features, the device 210 can also include a scented medium 219 applied to the first tip portion 216 and the second tip portion 215. The scented medium 219 can include, for example, eucalyptus, peppermint, tea tree oil and/or other scents that can provide a refreshing and invigorating cleansing experience for a user.

FIG. 3 is an isometric view of a disposable handheld device for manually cleaning a body orifice in accordance with another embodiment of the invention. The device 310 has a shaft 320 with an exterior surface 321. In the illustrated embodiment, the shaft 320 further includes a ridge portion 322 that extends outwardly from the exterior surface 321. The ridge portion 322 describes a generally helical path around the exterior surface 321 of the shaft 320.

In another aspect of this embodiment, a first tip portion 316 includes a first raised or ridge portion 318 that extends outwardly from an exterior surface 317. In this embodiment, the first ridge portion 318 describes a generally helical path around the exterior surface 317. In use, the first ridge portion 318 can engage and dislodge dust, debris, and other undesirable material from within a body orifice. In addition, the first tip portion 316 can absorb the dislodged material, thereby enabling the user to effectively clean the orifice. After use, the device 310 can be discarded.

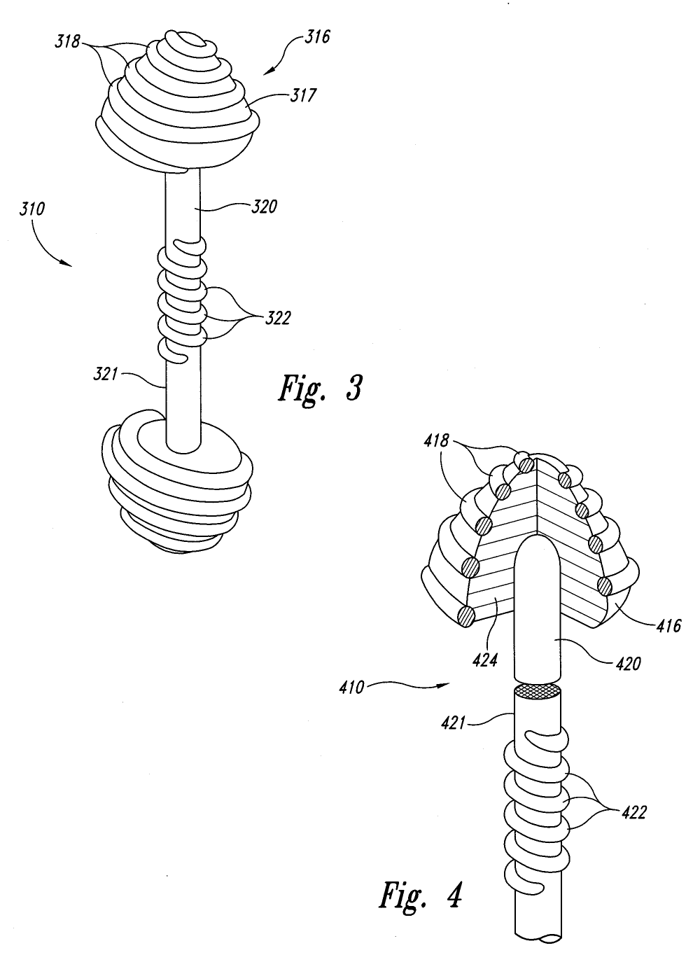

FIG. 4 is an exploded, partially cut-away isometric view of a portion of the cleaning device 310 of FIG. 3. In this embodiment, the portion 410 includes a shaft 420 with an exterior surface 421. The shaft 420 further includes a ridge portion 422 that extends outwardly from the exterior surface 421. In this embodiment, the tip portion 416 can be formed by compressing cotton, or another suitable material, in the desired conical shape. A suitable binder, e.g., a resin, can be used to hold the tip portion 416 in the desired shape after forming. In one embodiment, the ridge portion 418 may have a higher density than a body portion 424 of the tip portion 416. The higher density ridge portion 418 can provide a stiffer surface for engaging and dislodging material within a body orifice. The lower density body portion 424 can provide a softer surface for absorbing the dislodged material. In other embodiments, the tip portion 416 and the ridge portion 418 can be formed from other suitable materials, such as foam, synthetic fiber, organic material, etc.

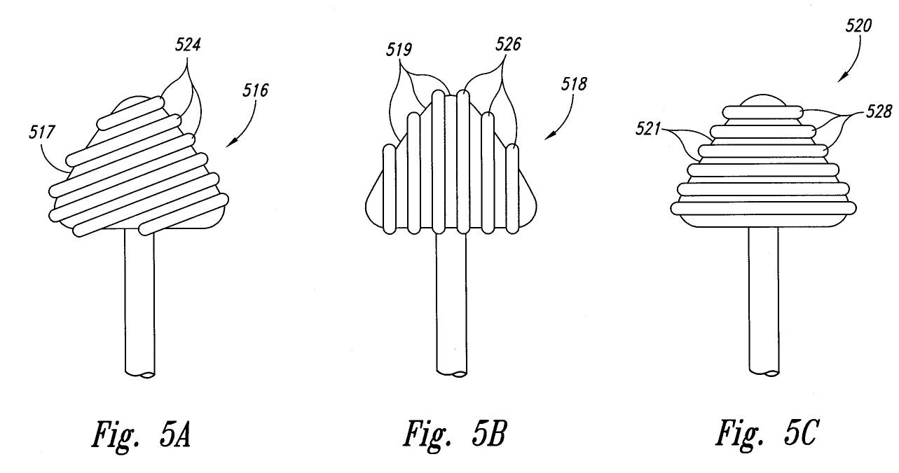

FIGS. 5A-5C are side views of device tip portions 516, 518, and 520, respectively, configured in accordance with embodiments of the invention. In FIG. 5A, a ridge portion 524 describes a generally helical path around an exterior surface 517 of the tip portion 516. In FIG. 5B, a plurality of ridge portions 526 describes generally vertical paths on an exterior surface 519 of the tip portion 518. In FIG. 5C, a plurality of ridge portions 528 describes generally horizontal paths on an exterior surface 521 of the tip portion 520. As discussed above, the ridge portions 524, 526, and 528 may be configured as shown in FIGS. 5A-5C to clean and/or remove unwanted material from within a nostril or other body orifice. One advantage of the different ridge configurations illustrated in FIGS. 5A-5C is that a user may find a certain configuration to be particularly well-suited to him or her for cleaning a certain body orifice. For example, a user may find the configuration of FIG. 5A to be especially suitable for cleaning a nostril, because the spiraling or helical ridge portion 524 may effectively dislodge and withdraw material within the nostril. As a further example, a user may find the configuration of FIG. 5C to be especially suitable for cleaning an ear canal and associated areas due to the characteristics of the horizontal ridge portion 528.

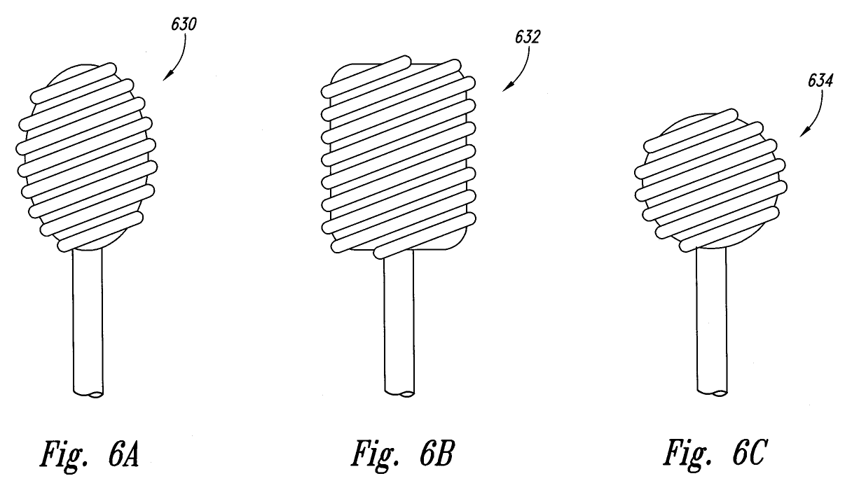

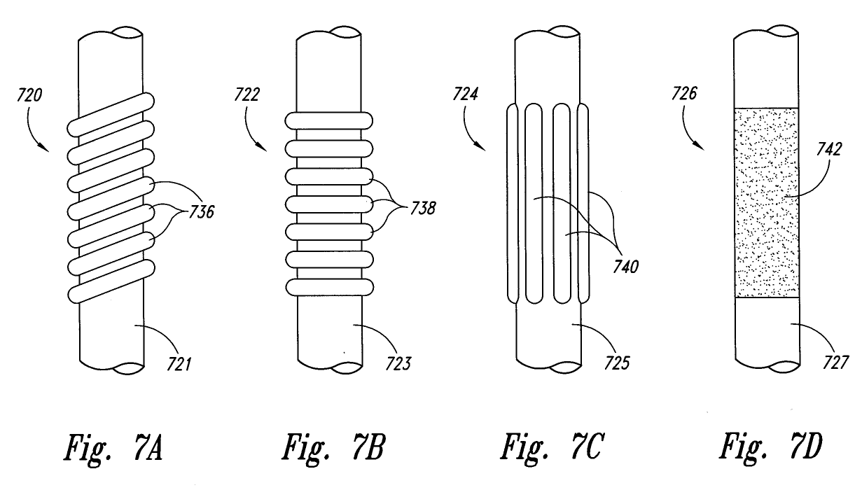

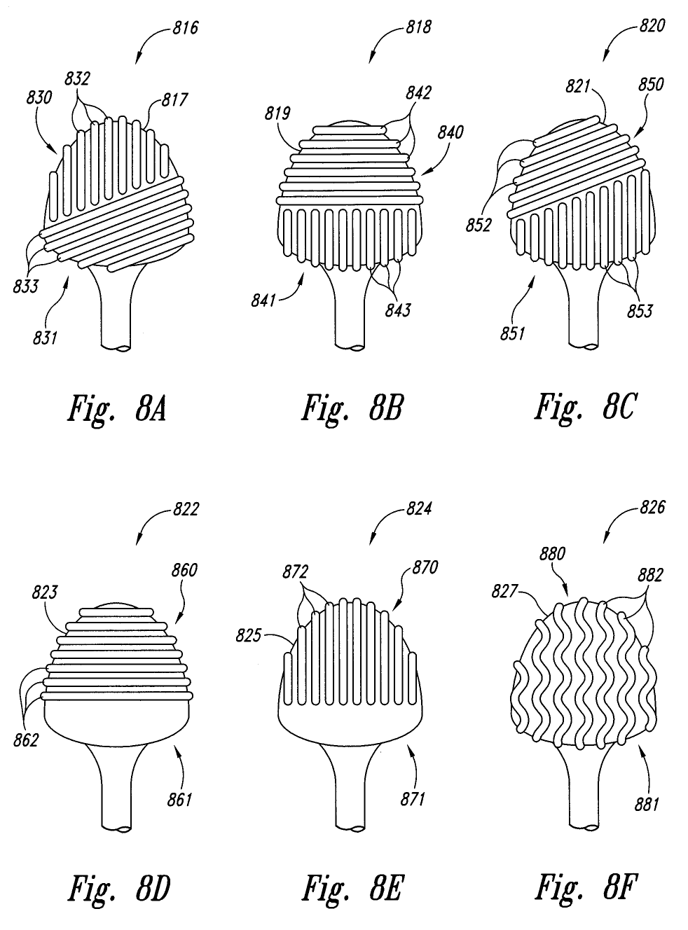

FIGS. 6A-6C are side views of device tip portions 630, 632, and 634, respectively, configured in accordance with other embodiments of the invention. In FIG. 6A, the tip portion 630 has a generally ellipsoidal shape. In FIG. 6B, the tip portion 632 has a generally cylindrical shape. In FIG. 6C, the tip portion 634 has a generally spherical shape.

The first and second tip portions of the device may be configured to have varying shapes as shown in FIGS. 6A-6C to meet differing cleaning needs. For example, one user may find that a device with a tip portion having a generally conical shape is well-suited for cleaning a nostril. Another user may find that a device with a tip portion having a generally ellipsoidal shape is well-suited for cleaning an ear canal. As those of ordinary skill in the art will appreciate, the present invention is not limited to the particular tip and/or shaft configuration described herein. Accordingly, a device configured in accordance with the present invention can have other useful shapes to meet personal body orifice cleaning needs.

The device tip portions can also have various sizes, such as small, medium, large, and extra-large, so as to meet the needs of different users. The device may also be sold in sets made up of one single shape and/or size along with a chart to enable a user to determine a preferred shape and/or size. The device may also be sold in a variety pack with multiple shapes and/or sizes, again along with a chart to enable a user to determine preferred shapes and/or sizes. One advantage of selling the personal cleaning device of the present invention in a variety pack is that it enables a user to choose from amongst the different shapes and or sizes. As described above, in some embodiments, the device includes a scented medium applied to the first and second tip portions. In some embodiments, the scented medium can include a medicinal herbal mixture of eucalyptus, peppermint, tea tree oil and other scents to provide a refreshing and invigorating cleansing experience. The scented medium can be non-toxic and non-allergenic, and can also include various ointments or lotions.

FIGS. 7A-7D are side views of device shaft portions 720, 722, 724, and 726, respectively, configured in accordance with embodiments of the invention. In FIG. 7A, a ridge portion 736 describes a generally helical path around an exterior surface 721 of the shaft portion 720. A user can grip the ridge portion 736 and rotate it to gradually insert the device into or out of a nostril or other body orifice. In FIG. 7B, a plurality of ridge portions 738 describes horizontal paths around an exterior surface 723 of the shaft portion 722. In FIG. 7C, a plurality of ridge portions 740 describes vertical paths around an exterior surface 725 of the shaft portion 724. In FIG. 7D, a ridge portion 742 may include multiple small ridges and/or other surface irregularities to provide a rough texture on the exterior surface 727 of the shaft portion 726. Or, the shaft may include multiple indentations to provide a rough texture on the exterior surface of the shaft.

The raised or ridge portions 736, 738, 740, and 742 enable a user to firmly grip the device while manually cleaning a body orifice. The shaft portions 720, 722, 724 and 726 may be made of various different materials such as wood, plastic, compressed paper and/or other suitable materials known in the art using a variety of suitable forming methods known in the art, such as tightly rolling die-cut paper. The raised or ridge portions 736, 738, 740 and 742 may also be made of a material such as wood, plastic, compressed paper, and/or other suitable materials known in the art. In various embodiments, the shaft portions 720, 722, 724 and 726 can be relatively rigid for favorable cleaning characteristics.

In some embodiments, the device may be manufactured by forming the shaft, and then compressing bodies of material to form the first and second tip portions. The ridge portion that extends outwardly from the exterior surface of the shaft may be integrally formed with the shaft. In some embodiments, the ridge portion on the shaft may be bonded to the shaft using a suitable adhesive.

FIGS. 8A-8F are side views of device tip portions 816, 818, 820, 822, 824, and 826, respectively, configured in accordance with embodiments of the invention. In FIG. 8A, the tip portion 816 includes an upper portion 830 and a lower portion 831. A plurality of ridge portions 832 describes generally vertical paths on an exterior surface 817 of the upper portion 830 of the tip portion 816. A ridge portion 833 describes a generally helical path around the exterior surface 817 of the lower portion 831 of the tip portion 816. In the configuration of FIG. 8A each of the upper portion 830 and the lower portion 831 constitutes approximately half of the tip portion 816. Other configurations (not shown) are equally foreseen, such as where the upper portion 830 constitutes more than half of the tip portion 816 and the lower portion constitutes less than half of the tip portion 816, or vice-versa.

In FIG. 8B, a plurality of ridge portions 842 describes generally horizontal paths on an exterior surface 819 of an upper portion 840 of the tip portion 818. A plurality of ridge portions 843 describes generally vertical paths on an exterior surface 819 of a lower portion 841 of the tip portion 818. In FIG. 8C, a ridge portion 852 describes a generally helical path around an exterior surface 821 of an upper portion 850 of the tip portion 820. A plurality of ridge portions 853 describes generally vertical paths on an exterior surface 821 of a lower portion 851 of the tip portion 820.

In the configurations illustrated in FIGS. 8D and 8E, the tip portions 822 and 824 include ridge portions 862 and 872 only on the exterior surfaces 823 and 825, of the upper portions 860 and 870, respectively. The exterior surfaces 823 and 825 of the lower portions 861 and 871, respectively, do not include ridge portions. In FIG. 8D, a plurality of ridge portions 862 describes generally horizontal paths on the exterior surface 823 of the upper portion 860 of the tip portion 822. In FIG. 8E, a plurality of ridge portions 872 describes generally vertical paths on the exterior surface 825 of the upper portion 870 of the tip portion 824.

In FIG. 8F, a plurality of ridge portions 882 describes generally curvilinear paths on an exterior surface 827 of the tip portion 826. In this configuration, each of the ridge portions 882 on the exterior surface 827 form multiple joined s-curves extending from an upper portion 880 of the tip portion 826 to a lower portion 881 of the tip portion 826.

The ridge portions 832, 833, 842, 843, 852, 853, 862, 872, and 882 may be configured as shown in FIGS. 8A-8F to clean and/or remove unwanted material from within a nostril or other body orifice. Other configurations (not shown) are equally foreseen. For example, a ridge portion can describe a helical path on an upper portion of a tip portion and a plurality of ridge portions can describe a horizontal path on a lower portion of the tip portion. Or, a ridge portion may be configured to describe a generally helical path only on an exterior surface of an upper portion of a tip portion. Or, ridge portions may be configured to describe generally helical, horizontal, or vertical paths only on an exterior surface of a lower portion of a tip portion. Ridge portions can be oriented in any fashion on an upper portion of a tip portion and in any other fashion on a lower portion of a tip portion. One advantage of the different ridge portion configurations illustrated in FIGS. 8A-8F is that a user may find a certain configuration to be particularly well-suited to him or her for cleaning a certain body orifice. For example, a user may find the configurations of FIGS. 8A-8C to be especially suitable for cleaning a particular body orifice due to the different orientations of the ridge portions on the upper and lower portions of the tip portion. As another example, a user may find the configurations of FIGS. 8D-8E to be especially suitable for cleaning a different body orifice due to the presence of ridge portions only on the upper portions of the tip portions. As a further example, a user may find the configuration of FIG. 8F to be especially suitable for effectively dislodging and withdrawing material within the nostril due to the characteristics of the curvilinear ridge portions 882. Another advantage of the configurations of FIGS. 8A-8F is that they can offer more choices to users, either when sold in sets of individual configurations or in variety packs as described above.

From the foregoing, it will be appreciated that specific embodiments of the invention have been described herein for purposes of illustration, but that various modifications may be made without deviating from the invention. For example, in some embodiments, the ridge portions that extend outwardly from the exterior surfaces of the first and second tip portions may describe paths other than a helical, vertical, or horizontal path around the exterior surfaces of the first and second tip portions. Aspects of the invention described in the context of particular embodiments may be combined or eliminated in other embodiments. For example, the shaft of the device may not have a ridge portion. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited, except as by the appended claims.

I/We claim:

1. A disposable handheld device for manually cleaning a body orifice, the device comprising:
a shaft having:
a first end portion;
a second end portion spaced apart from the first end portion to define a first exterior surface portion therebetween, the first exterior surface portion having a first diameter; and
a first ridge portion extending outwardly from the first exterior surface portion;
a first tip portion positioned toward the first end portion of the shaft, the first tip portion having:
a second exterior surface portion defining a first generally conical shape having a second diameter greater than the first diameter; and
a second ridge portion extending outwardly from the second exterior surface portion; and
a second tip portion positioned toward the second end portion of the shaft;
the second tip portion having:
a third exterior surface portion defining a second generally conical shape having a third diameter greater than the first diameter; and
a third ridge portion extending outwardly from the third exterior surface portion.

2. The disposable handheld device of claim 1 wherein the second ridge portion describes a generally helical path around the second exterior surface portion and the third ridge portion describes a generally helical path around the third exterior surface portion.

3. The disposable handheld device of claim 1 wherein the first ridge portion describes a generally helical path around the first exterior surface portion,

4. The disposable handheld device of claim 1, further comprising a scented medium applied to the first and second tip portions.

5. The disposable handheld device of claim 4 wherein the scented medium includes an herbal scent.

6. The disposable handheld device of claim 1 wherein the first tip portion includes a first cotton body and the second tip portion includes a second cotton body.

7. The disposable handheld device of claim 1 wherein the first tip portion includes a first foam body and the second tip portion includes a second foam body.

8. A disposable handheld device for manually cleaning a body orifice, the device comprising:
a shaft comprising a first end portion spaced apart from a second end portion; and
a tip portion positioned toward the first end portion of the shaft, the tip portion including:
an exterior surface; and
a ridge portion extending outwardly from the exterior surface.

9. The disposable handheld device of claim 8 wherein the tip portion is a first tip portion, the exterior surface is a first exterior surface, the ridge portion is a first ridge portion, and further comprising a second tip portion positioned toward the second end portion of the shaft, the second tip portion including:
a second exterior surface; and
a second ridge portion extending outwardly from the second exterior surface.

10. The disposable handheld device of claim 9 wherein the shaft further comprises:
a third exterior surface between the first and second end portions; and
a plurality of third ridge portions extending outwardly from the third exterior surface.

11. The disposable handheld device of claim 9 wherein the shaft further comprises:
a third exterior surface between the first and second end portions; and
a third ridge portion that extends outwardly from the third exterior surface.

12. The disposable handheld device of claim 9 wherein the shaft defines a first cross-sectional dimension, the first exterior surface has a first shape defining a second cross-sectional dimension greater than the first cross-sectional dimension, and the second exterior surface has a second shape defining a third cross-sectional dimension greater than the first cross-sectional dimension.

13. The disposable handheld device of claim 12 wherein the first and second shapes are at least generally conical.

14. The disposable handheld device of claim 12 wherein the first and second shapes are at least generally cylindrical.

15. The disposable handheld device of claim 12 wherein the first and second shapes are at least generally ellipsoidal.

16. The disposable handheld device of claim 9 wherein the first ridge portion describes a generally helical path around the first exterior surface and the second ridge portion describes a generally helical path around the second exterior surface.

17. The disposable handheld device of claim 9 wherein the first ridge portion describes a generally horizontal path around the first exterior surface.

18. The disposable handheld device of claim 9 wherein the first ridge portion describes a generally vertical path around the first exterior surface.

19. The disposable handheld device of claim 9 wherein the first ridge portion describes a generally curvilinear path around the first exterior surface.

20. The disposable handheld device of claim 9, further comprising a medicinal medium applied to the first tip portion and the second tip portion.

21. The disposable handheld device of claim 9 wherein the first tip portion and the second tip portion include a first material having a first density, and wherein the first ridge portion and the second ridge portion include a second material having a second density that is greater than the first density.

22. The disposable handheld device of claim 9 wherein the first tip portion includes an upper portion and a lower portion, the first exterior surface extends over both the upper portion and the lower portion, the first ridge portion extends outwardly from the first exterior surface extending over the upper portion and further comprising a third ridge portion extending outwardly from the first exterior surface extending over the lower portion.

23. The disposable handheld device of claim 22 wherein the first ridge portion and the third ridge portion describe different orientations around the first exterior surface.

24. A personal hygiene device comprising:
first means for engaging debris within a body orifice, the first means including:
a first exterior surface; and
a first raised portion extending outwardly from the first exterior surface;
second means for engaging debris within the body orifice, the second means comprising:
a second exterior surface; and
a second raised portion extending outwardly from the second exterior surface; and
means for operably coupling the first and second means to opposing end portions of a manipulatable handle.

25. The disposable handheld device of claim 24 wherein the handle includes a third exterior surface with means for gripping the handle that extend outwardly from the third exterior surface.

26. The disposable handheld device of claim 24 wherein the first and second means include aromatic means for providing a scent.
